(19) United States
(12) Patent Application Publication
(10) Pub. No.: US 2019/0058819 A1
(43) Pub. Date: Feb. 21, 2019

(54) ENDOSCOPE APPARATUS

(71) Applicant: Sony Olympus Medical Solutions Inc., Tokyo (JP)

(72) Inventor: Motoaki Kobayashi, Tokyo (JP)

(73) Assignee: Sony Olympus Medical Solutions Inc., Tokyo (JP)

(21) Appl. No.: 16/034,521

(22) Filed: Jul. 13, 2018

(30) Foreign Application Priority Data
Aug. 18, 2017 (JP) 2017-158169

(51) Int. Cl. H04N 5/238; G02B 23/24; G02B 27/00; A61B 1/06; A61B 1/00; A61B 1/04

(52) U.S. Cl. CPC H04N 5/238; G02B 23/2484; G02B 23/2446; A61B 1/04; A61B 1/0661; A61B 1/00006; G02B 27/0075

(57) ABSTRACT

An endoscope apparatus is disclosed which includes an imaging device to which plural kinds of endoscopes having optical systems with different aperture diameters are connectable, the imaging device including: an aperture stop with a light transmission region that allows light to be transmitted to an endoscope among the plural kinds of endoscopes that is connected to the imaging device, wherein a size of the light transmission region is changeable; and an imaging unit receiving the light transmitted through the aperture stop and converting the light into an electric signal; an aperture diameter determining unit that determines an aperture diameter of the connected endoscope, based on an image generated by the electric signal generated by the imaging device; and a control unit that determines the size of the light transmission region, based on the aperture diameter determined by the aperture diameter determining unit, and changes the light transmission region.

### CROSS-REFERENCE TO RELATED APPLICATION(S)

The present application claims priority to and incorporates by reference the entire contents of Japanese Patent Application No. 2017-158169 filed in Japan on Aug. 18, 2017.

### BACKGROUND

The present disclosure relates to an endoscope apparatus.

In the past, an endoscope apparatus has been known which observes an inside of a subject such as a person and a mechanical structure in a medical field and an industrial field, respectively (for example, Japanese Laid-open Patent Publication No. 2015-134039 A). The endoscope apparatus disclosed in the above publication includes an endoscope which is inserted into a subject and acquires a subject image inside the subject from a distal end, an imaging element (an image sensor) which is equipped with the endoscope and is used to capture a subject image and to output an image signal, a control device which generates a display video signal by processing the image signal, and a display device which displays an image based on the display video signal.

In recent years, the number of pixels of the image sensor has been increased in order to improve the image resolution. At this time, the depth of field also becomes shallow as a stop value decreases due to an increase in the number of pixels. As a result, because the depth of field becomes shallow even when the resolution of the captured image is improved, an observation becomes difficult depending on a subject.

As a technique of deepening the depth of field, a technique of increasing the depth of field by decreasing an aperture diameter of a stop is known. In this technique, it is possible to adjust the depth of field by adjusting the aperture diameter the stop.

### SUMMARY

An imaging device can be equipped with various endoscopes having different optical characteristics. For example, various endoscopes having different aperture diameters in the optical systems are attached. When the aperture diameter of the optical system is different, the aperture diameter of the stop for enlarging the depth of field is also different.

The present disclosure has been made in view of the above, and is directed to an improvement to an endoscope apparatus.

According to an aspect of the present disclosure, an endoscope apparatus is provided which includes an imaging device to which a plurality of kinds of endoscopes having optical systems with different aperture diameters are connectable, the imaging device including: an aperture stop with a light transmission region that allows light to be transmitted to an endoscope among the plurality of kinds of endoscopes that is connected to the imaging device, wherein a size of the light transmission region is changeable; and an imaging unit that receives the light transmitted through the aperture stop and converts the light into an electric signal; an aperture diameter determining unit that determines an aperture diameter of the endoscope connected to the imaging device, based on an image generated by the electric signal generated by the imaging device; and a control unit that determines the size of the light transmission region formed by the aperture stop, based on the aperture diameter determined by the aperture diameter determining unit, and changes the light transmission region of the aperture stop.

The above and other objects, features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a schematic configuration of an endoscope apparatus according to a first embodiment of the disclosure;

FIG. 2 is a block diagram illustrating a configuration of a camera head and a control device illustrated in FIG. 1;

FIG. 3A is a schematic diagram illustrating a configuration an endoscope and a camera head according to the first embodiment of the disclosure;

FIG. 3B is a schematic diagram illustrating a configuration of the endoscope and the camera head according to the first embodiment of the disclosure;

FIG. 4 is a diagram illustrating an aperture stop of the endoscope according to the first embodiment of the disclosure;

FIG. 5 is a diagram illustrating an example of an image captured by the camera head according to the first embodiment of the disclosure;

FIG. 6 is a diagram illustrating an example of an image captured by the camera head according to the first embodiment of the disclosure;

FIG. 7 is a diagram illustrating an aperture stop of the endoscope according to the first embodiment of the disclosure;

FIG. 8 is a flowchart illustrating a process that is performed by the endoscope apparatus according to the first embodiment of the disclosure;

FIG. 9 is a flowchart illustrating a process that is performed by an endoscope apparatus according to a second embodiment of the disclosure;

FIG. 10 is a diagram illustrating an example of an image captured by a camera head according to the second embodiment of the disclosure;

FIG. 11 is a diagram illustrating an aperture stop of an endoscope according to the second embodiment of the disclosure;

FIG. 12 is a flowchart illustrating a process that is performed by an endoscope apparatus according to a modified example of the second embodiment of the disclosure;

FIG. 13 is a schematic diagram illustrating a configuration of an endoscope and a camera head according to a third embodiment of the disclosure;

FIG. 14 is a diagram illustrating an aperture stop of the endoscope according to the third embodiment of the disclosure;

FIG. 15 is a diagram illustrating the aperture stop as viewed from a direction A of FIG. 14; and

FIG. 16 is a diagram illustrating the aperture stop as viewed from a direction B of FIG. 14.

### DETAILED DESCRIPTION

Hereinafter, a mode for carrying out the disclosure (hereinafter, referred to as an “embodiment”) will be described. In the embodiment, a medical endoscope apparatus that captures and displays an image inside a subject such as a patient will be described as an example of an endoscope apparatus according to the disclosure. Further, the disclosure is not limited to the embodiment. In the description of the drawings, a description will be made by giving the same reference numerals to the same components.

### First Embodiment

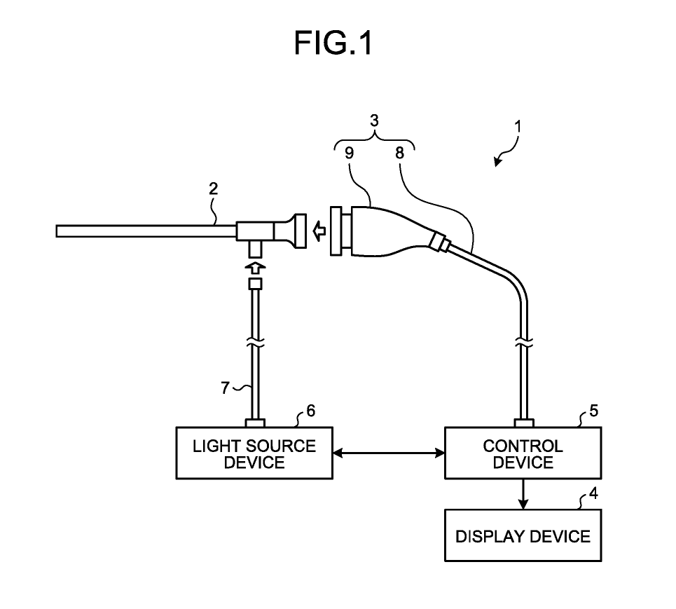

FIG. 1 is a diagram illustrating a schematic configuration of an endoscope apparatus 1 according to a first embodiment of the disclosure. The endoscope apparatus 1 is an apparatus that is used in a medical field and observes a subject inside an observation object such as a person (a living body). The endoscope apparatus 1 includes, as illustrated in FIG. 1, an endoscope 2, an imaging device 3 (a medical imaging device), a display device 4, a control device 5 (an image processing device), and a light source device 6. A medical image acquiring system is constituted by the imaging device 3 and the control device 5. Note that the endoscope 2 and the imaging device 3 constitute an endoscope apparatus using a rigid endoscope in the first embodiment.

The light source device 6, to which one end of a light guide 7 is connected, supplies white illumination light for illuminating the inside of the living body to the one end of the light guide 7. While one end of the light guide 7 is detachably connected to the light source device 6, the other end thereof is detachably connected to the endoscope 2. Then, the light guide 7 transmits the light supplied from the light source device 6 from one end to the other end thereof, and supplies the light to the endoscope 2.

The imaging device 3 captures a subject image from the endoscope 2 and outputs the imaging result. The imaging device 3 includes, as illustrated in FIG. 1, a camera head 9 and a transmission cable 8 which is a signal transmission portion. In the first embodiment, the transmission cable 8 and the camera head 9 constitute a medical imaging device.

The endoscope 2 of rigid type has an elongated shape and is inserted into the living body. Inside the endoscope 2, an optical system is provided which includes one or more lenses and collects a subject image. The endoscope 2 emits the light supplied via the light guide 7 from the distal end so that the inside of the living body is irradiated with the light. Then, the light irradiated into the living body (the subject image) is collected by the optical system inside the endoscope 2.

The camera head 9 is detachably connected to a proximal end of the endoscope 2. Then, the camera head 9 captures a subject image collected in the endoscope 2 under the control of the control device 5, and outputs an imaging signal by the capturing operation. The camera head 9 will be described in detail later.

One end of the transmission cable 8 is detachably connected to the control device 5 via a connector and the other end thereof is detachably connected to the camera head 9 via a connector. Specifically, the transmission cable 8 is a cable in which a plurality of electric wires (not illustrated) are disposed at the inside of the outer sheath corresponding to the outermost layer. The plurality of electric wires are electric wires for transmitting imaging signals output from the camera head 9 and control signals, synchronization signals, clocks, and electric power output from the control device 5 to the camera head 9.

The display device 4 displays an image generated by the control device 5 under the control of the control device 5. In order to easily produce a sense of immersion at the time of observation, it is desirable that the display device 4 includes a display unit of, for example but not limited to, 55 inches or more.

The control device 5 processes an imaging signal input from the camera head 9 via the transmission cable 8, outputs the image signal to the display device 4, and comprehensively controls the operations of the camera head 9 and the display device 4. The control device 5 will be described in detail later.

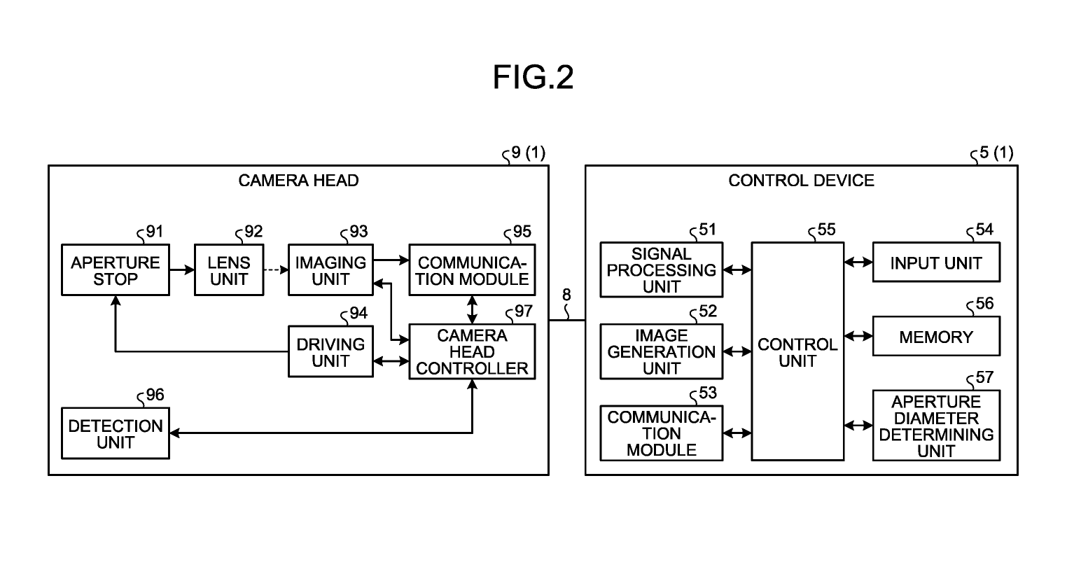

Next, a configuration of the imaging device 3 and the control device 5 will be described. FIG. 2 is a block diagram illustrating a configuration of the camera head 9 and the control device 5. Note that, in FIG. 2, a connector for detachably connecting the camera head 9 and the transmission cable 8 to each other is not illustrated.

Hereinafter, a configuration of the control device 5 and a configuration of the camera head 9 will be sequentially described. Note that in the description below, a main part of the first embodiment will be chiefly described as the configuration of the control device 5. The control device 5 includes, as illustrated in FIG. 2, a signal processing unit 51, an image generation unit 52, a communication module 53, an input unit 54, a control unit 55, a memory 56, and an aperture diameter determining unit 57. Note that the control device 5 may be provided with a power supply unit which generates a power voltage for driving the control device 5 and the camera head 9, supplies the power voltage to each of units of the control device 5, and supplies the power voltage to the camera head 9 via the transmission cable 8.

The signal processing unit 51 outputs a digital image signal (RAW signal) to the image generation unit 52 by performing a noise reduction process or a signal process such as A/D conversion if necessary on the imaging signal output from the camera head 9.

Further, the signal processing unit 51 generates a synchronization signal and a clock for the imaging device 3 and the control device 5. The synchronization signal (for example, a synchronization signal for instructing the imaging timings of the camera head 9) or the clock (for example, a serial communication clock) for the imaging device 3 are sent to the imaging device 3 by a line (not illustrated), and the imaging device 3 is driven based on the synchronization signal or the clock.

The image generation unit 52 generates a display image signal, which is displayed by the display device 4, based on the imaging signal input from the signal processing unit 51. The image generation unit 52 generates a display image signal including a subject image by performing a predetermined signal process on the imaging signal. Here, the image generation unit 52 generates a captured image by performing known image processes corresponding to various image processes such as an interpolation process, a color correction process, and a noise reduction process as the image process. The image generation unit 52 outputs the generated display image signal to the display device 4.

Specifically, the image generation unit 52 multiplies the image signal (RAW signal (digital signal)) by a digital gain for amplifying the digital signal. Further, the image generation unit 52 performs a RAW process such as an optical black subtraction process and a demosaic process on the image signal (RAW signal (digital signal)) multiplied by the digital gain, and converts the RAW signal (image signal) into an RGB signal (image signal). Further, the image generation unit 52 performs an RGB process such as white balance adjustment of multiplying an RGB value by a gain, RGB gamma correction, and YC conversion (conversion from an RGB signal into a luminance signal and a color difference signal (Y/C_{b}/Cr signals)) on the RGB signal (image signal). Further, the image generation unit 52 performs an YC process such as color difference correction and noise reduction on the Y/C_{b}/Cr signals (image signals).

The communication module 53 outputs a signal from the control device 5 to the imaging device 3 (FIG. 1). This signal includes the control signal, which will be described later, transmitted from the control unit 55. The communication module 53 outputs a signal (for example, an image signal) from the imaging device 3 to the control device 5. That is, the communication module 53 is a relay device which collectively outputs signals generated from the components of the control device 5 and output to the imaging device 3 in terms of, for example, parallel/serial conversion or the like and outputs signals input from the imaging device 3 to the components of the control device 5 by distributing the signals in terms of, for example, serial/parallel conversion or the like.

The input unit 54 is realized by using a user interface such as a keyboard, a mouse, and a touch panel and receives various kinds of information.

The control unit 55 performs driving controls of the components including the control device 5 and the camera head 9 and input and output controls of information with respect to the components. The control unit 55 generates a control signal by referring to communication information data (for example, communication format information or the like) stored in the memory 56 and transmits the generated control signal to the imaging device 3 via the communication module 53. Further, the control unit 55 outputs the control signal to the camera head 9 via the transmission cable 8.

The memory 56 is realized by using a semiconductor memory such as a flash memory or DRAM (Dynamic Random Access Memory) and stores communication information data (for example, communication formation information or the like). In addition, the memory 56 may store various programs to be executed by the control unit 55.

The aperture diameter determining unit 57 determines the aperture diameter of the optical system of the endoscope 2 connected to the camera head 9 based on an optical image projected on an image generated by the image generation unit 52. The aperture diameter determining unit 57 performs an aperture diameter determining process by using an image signal subjected to a white balance adjustment process performed by the image generation unit 52, for example, when the endoscope 2 is connected to the camera head 9. The aperture diameter determining process will be described later.

Note that the signal processing unit 51 may include an AF processing unit which outputs a predetermined AF evaluation value for each frame based on the imaging signal of the input frame and an AF calculating unit which performs an AF calculating process of selecting a frame or focus lens position most suitable for a focus position from the AF evaluation value of each frame obtained from the AF processing unit.

The signal processing unit 51, the image generation unit 52, the communication module 53, the control unit 55, and the aperture diameter determining unit 57 are realized by using a general purpose processor such as a central processing unit (CPU) having an internal memory (not illustrated) storing a program or a dedicated processor such as various calculation circuits for performing a specific function such as an application specific integrated circuit (ASIC). Further, these components may be configured by using field programmable gate array (FPGA: not illustrated) which is a kind of programmable integrated circuit. Also, in the case of the configuration of FPGA, a memory storing configuration data may be provided and FPGA corresponding to a programmable integrated circuit may be configured by the configuration data read from the memory.

Next, explanations will be made chiefly about a main part of the first embodiment, regarding the camera head 9. The camera head 9 includes, as illustrated in FIG. 2, an aperture stop 91, a lens unit 92, an imaging unit 93, a driving unit 94 (FIG. 1), a communication module 95, a detection unit 96, and a camera head controller 97.

The aperture stop 91 is disposed at a position through which the optical axis of the camera head 9 passes and which corresponds to an entrance pupil position of the lens unit 92. The aperture stop 91 is configured by using a liquid crystal. Specifically, the aperture stop 91 is formed of two glass plates bonded to each other and a liquid crystal enclosed therein, and thus has a plate shape. The aperture stop 91 can form a region in which light is transmitted (hereinafter, referred to as a light transmission region) and a region in which light is shielded (hereinafter, referred to as a light shield area) in accordance with the orientation of the liquid crystal. The aperture stop 91 which is configured by using such a liquid crystal can change the position and the size of the light transmission region by changing the orientation of the liquid crystal under the control of the driving unit 94. Note that the optical axis of the camera head 9 passes through the center of the light receiving surface of the image sensor of the imaging unit 93 and extends in a direction orthogonal to the light receiving surface.

The lens unit 92 is configured by using one or more lenses, and forms a subject image passing through the aperture stop 91 on an imaging surface of the image sensor constituting the imaging unit 93. The one or more lenses are movable along the optical axis. Then, the lens unit 92 is provided with an optical zoom mechanism (not illustrated) which changes a viewing angle and/or a focus mechanism which changes a focus position by moving the one or more lenses. Additionally, the lens unit 92 may be provided with an optical filter (for example, a filter that cuts off infrared light) or the like which is insertable and removable on the optical axis, other than the optical zoom mechanism and the focus mechanism.

The imaging unit 93 captures an image of a subject under the control of the camera head controller 97. The imaging unit 93 is configured by using an image sensor which receives a subject image formed by the lens unit 92, and converts the subject image into an electric signal. The image sensor is configured as a charge coupled device (CCD) image sensor or a complementary metal oxide semiconductor (CMOS) image sensor. When the image sensor is the CCD, for example, a signal processing unit (not illustrated) may be mounted on a sensor chip or the like. The signal processing unit may perform a signal process (A/D conversion or the like) on the electric signal (analog signal) from the image sensor and outputs an imaging signal. When the image sensor is the CMOS, the image sensor includes the signal processing unit which performs a signal process (A/D conversion or the like) on the electric signal (analog signal) converted from, for example, light and outputs an imaging signal. The imaging unit 93 outputs the generated electric signal to the communication module 95.

The driving unit 94 performs driving control of forming the light transmission region and the light shield region by controlling the orientation of the liquid crystal of the aperture stop 91 in response to the observation mode under the control of the camera head controller 97. Further, the driving unit 94 may include a driver which changes the viewing angle or the focus position of the lens unit 92 by operating the optical zoom mechanism or the focus mechanism.

The communication module 95 outputs the signal transmitted from the control device 5 to the components inside the camera head 9 such as the camera head controller 97. Further, the communication module 95 converts information on the current state of the camera head 9 into a signal format in accordance with a predetermined transmission method, and outputs the converted signal to the control device 5 via the transmission cable 8. That is, the communication module 95 is a relay device which outputs the signals that have been input from the control device 5 or the transmission cable 8 to the components of the camera head 9 in accordance with, for example, serial/parallel conversion or the like, and outputs the signals from the components of the camera head 9 to the control device 5 or the transmission cable 8 in accordance with, for example, parallel/serial conversion or the like.

The detection unit 96 detects whether or not the endoscope 2 is connected to the camera head 9. The detection unit 96 detects whether or not the endoscope 2 is connected to the camera head 9 by using a known detection mechanism, for example, a mechanical detection mechanism such as a button or an optical detection mechanism using infrared light or the like.

The camera head controller 97 controls the entire operations of the camera head 9 in response to a driving signal input via the transmission cable 8 or an instruction signal output from a manipulation unit by a user's manipulation to the operating unit such as a switch provided on an outer surface of the camera head 9 in an exposed state. Further, the camera head controller 97 outputs information on the current state of the camera head 9 to the control device 5 via the transmission cable 8.

Note that the driving unit 94, the communication module 95, the detection unit 96, and the camera head controller 97 are realized by using a general purpose processor such as a CPU having an internal memory (not illustrated) storing a program, or a dedicated processor such as various calculation circuits for performing a specific function such as ASIC. Further, these components may be configured by using FPGA which is a kind of programmable integrated circuit. Also, in the case of the configuration of FPGA, a memory storing configuration data may be provided and FPGA corresponding to a programmable integrated circuit may be configured by the configuration data read from the memory.

Additionally, the camera head 9 or the transmission cable 8 may be provided with a signal processing unit which performs a signal process on the imaging signal generated by the communication module 95 or the imaging unit 93. Further, based on a reference clock generated by an oscillator (not illustrated) provided inside the camera head 9, an imaging clock for driving the imaging unit 93 and a driving clock for driving the driving unit 94 may be generated and output to the imaging unit 93 and the driving unit 94. Then, based on the synchronization signal input from the control device 5 via the transmission cable 8, various process timing signals of the imaging unit 93, the driving unit 94, and the camera head controller 97 may be generated and output to the imaging unit 93, the driving unit 94, and the camera head controller 97. Further, the camera head controller 97 may be provided in the transmission cable 8 or the control device 5 instead of the camera head 9.

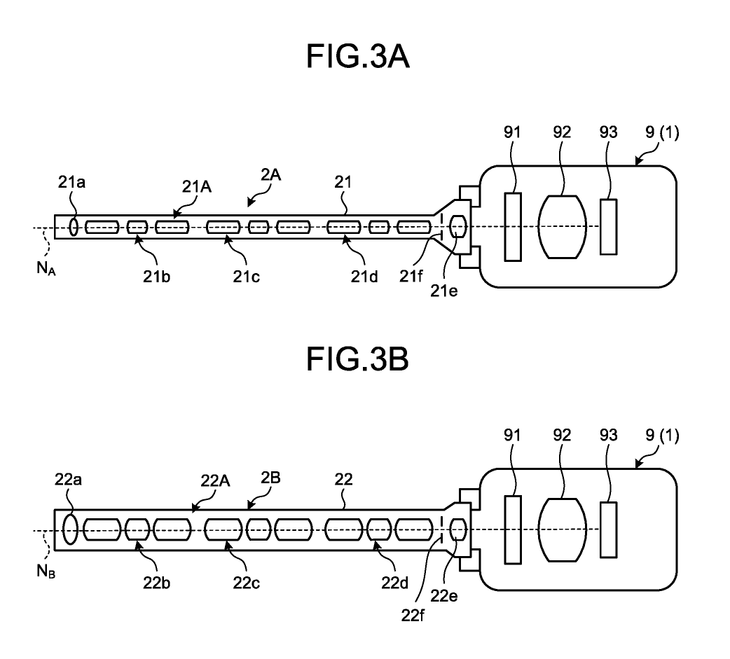

FIGS. 3A and 3B are schematic diagrams illustrating a configuration of the endoscope 2 and the camera head 9 according to the embodiment of this disclosure. FIGS. 3A and 3B are diagrams illustrating a state where the endoscope 2 and the camera head 9 illustrated in FIG. 1 are rotated by 90° using a longitudinal axis as a rotation axis. As the endoscope 2 attached to the camera head 9, there exist endoscopes 2A and 2B illustrated in FIGS. 3A and 3B, respectively. The endoscopes 2A and 2B receive external light at the distal end side and output the light to the camera head 9 at the proximal end side. In the endoscopes 2A and 2B, the optical systems have different aperture diameters. In the first embodiment, an optical axis N_{A} of the endoscope 2A coincides with the optical axis of the camera head 9 and an optical axis N_{B} of the endoscope 2B coincides with the optical axis of the camera head 9.

The endoscope 2A includes an optical system 21A inside an insertion portion 21. In the optical system 21A, an objective lens 21a, a first relay optical system 21b, a second relay optical system 21c, a third relay optical system 21d, and an eyepiece 21e are arranged in this order from the distal end side along the optical axis N_{A} of the optical system 21A. Further, the endoscope 2A is provided with a mask 21f having a circular opening corresponding to the aperture diameter of the optical system 21A.

In the endoscope 2B, the diameter of an insertion portion 22 is larger than the diameter of the insertion portion 21 of the endoscope 2A. The endoscope 2B includes an optical system 22A inside the insertion portion 22. In the optical system 22A, an objective lens 22a, a first relay optical system 22b, a second relay optical system 22c, a third relay optical system 22d, and an eyepiece 22e are arranged in this order from the distal end side along the optical axis N_{B} of the optical system 22A. The aperture diameter of the optical system 22A is larger than the aperture diameter of the optical system 21A of the endoscope 2A. The aperture diameter mentioned herein indicates a diameter of a portion through which light passes in each optical system. Further, the endoscope 2B is provided with a mask 22f having a circular opening corresponding to the aperture diameter of the optical system 22A.

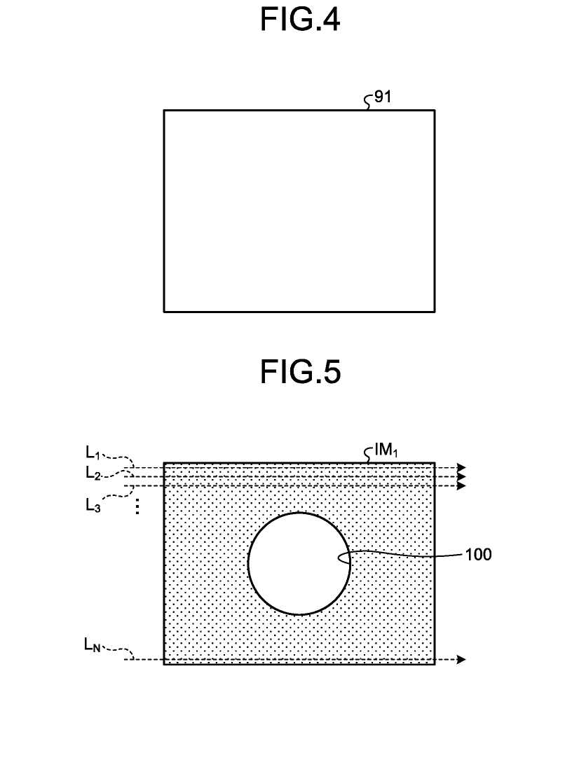

Next, a depth enlargement process according to the aperture stop 91 will be described with reference to FIGS. 4 to 7. FIG. 4 is a diagram illustrating the aperture stop 91 of the endoscope 2 according to the first embodiment of the disclosure. FIG. 4 illustrates an example of a state in which all regions capable of transmitting or shielding light in the aperture stop 91 are light transmission regions (hereinafter, this state will be referred to as a total transmission state).

First, a process of detecting the aperture diameter of the connected endoscope 2 will be described. When the camera head 9 is activated and the connection of the endoscope 2 to the camera head 9 is detected the camera head 9, the endoscope apparatus 1 controls the aperture stop 91 so as to be in the total transmission state (see FIG. 4) and receives observation light from the endoscope 2 so that the optical image is captured as an image.

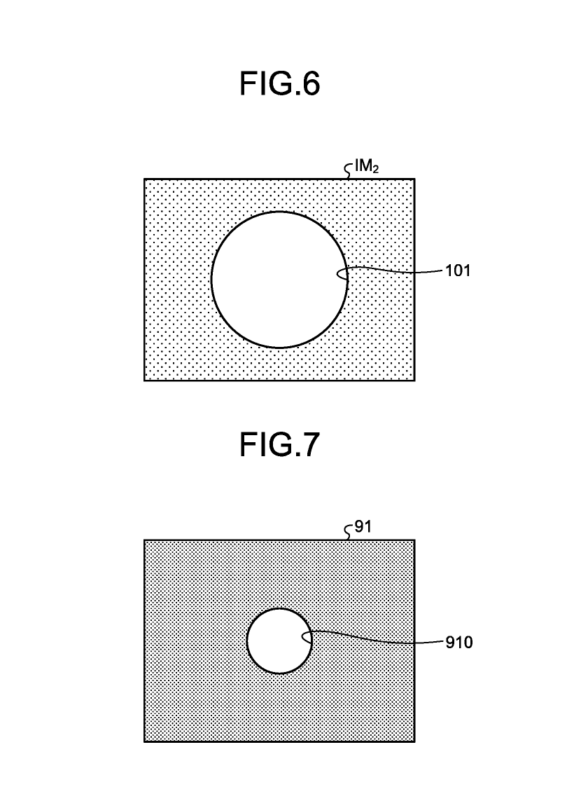

FIGS. 5 and 6 are diagrams illustrating examples of images captured by the camera head 9 according to the first embodiment of the disclosure. When the endoscope 2, for example, the endoscope 2A is connected to the camera head 9 and the aperture stop 91 is in the total transmission state, an image (mask image 100) is projected like an image IM₁ illustrated in FIG. 5 by the light passing through the mask 21f provided depending on the aperture diameter of the optical system 21A. In this embodiment, a description will be made on the assumption that the mask has a circular opening. However, the shape of the opening of the mask may be an oval or polygonal shape other than the circle in other embodiments.

Meanwhile, when the endoscope 2B is connected to the camera head 9 and the aperture stop 91 is in the total transmission state, like an image IM₂ illustrated in FIG. 6, an image (mask image 101) is projected by the light passing through the mask 21f provided depending on the aperture diameter of the optical system 22A. The diameter of the mask image 101 is larger than that of the mask image 100 obtained when the endoscope 2A is connected.

The aperture diameter determining unit 57 acquires a luminance signal (Y signal) from the image signals (Y/C_{b}/Cr signals) processed by the image generation unit 52 for, for example, the image IM₁ illustrated in FIG. 5. Then, the aperture diameter determining unit 57 detects a distribution of luminance values of a plurality of horizontal lines L₁, L₂, L₃, . . . L_{N} (N is a natural number) inside the image IM₁ based on the luminance signal (Y signal). Here, in the image IM₁, the luminance value of the region of the mask image 100 is higher than that of the other regions. That is, in the luminance distribution of the horizontal line including the mask image 100, a difference in luminance value between two boundary points of the region of the mask image 100 and the other region increases. For this reason, the aperture diameter determining unit 57 compares the luminance value on the horizontal line with a threshold value, and recognizes a region in which pixels having a luminance value higher than the threshold value are arranged as a part of the mask image 100. The aperture diameter determining unit 57 recognizes the mask image 100 as the entire image by performing the above-described process on all horizontal lines L₁, L₂, L₃, . . . L_{N}. Then, the aperture diameter determining unit 57 calculates the diameter of the circle formed by the outer edge of the recognized mask image 100.

The aperture diameter determining unit 57 determines the aperture diameter of the optical system of the connected endoscope 2 based on the calculated diameter of the circle. Specifically, the aperture diameter determining unit 57 determines the aperture diameter from the calculated diameter of the circle, for example, by referring to the information illustrating a relationship between the aperture diameter and the diameter of the circle stored in the memory 56. In this way, the aperture diameter determining unit 57 determines the aperture diameter of the connected endoscope 2 from the captured image.

Then, the control unit 55 determines the diameter of the light transmission region of the aperture stop 91 depending on the aperture diameter determined by the aperture diameter determining unit 57, generates a control signal for forming the light transmission region having a determined diameter in the aperture stop 91, and outputs the control signal to the camera head controller 97.

FIG. 7 is a diagram illustrating the aperture stop 91 of the endoscope 2 according to the first embodiment of the disclosure. When the camera head controller 97 acquires the control signal for the aperture stop 91 from the control unit 55 and acquires a control signal indicating the depth enlargement mode setting is input, a light transmission region 910 having a diameter determined by the control unit 55 is formed in the aperture stop 91 in accordance with the control signals (see FIG. 7). At this time, a region other than the light transmission region 910 becomes the light shield region. Accordingly, because the light transmission region corresponding to the aperture diameter of the connected endoscope 2 is formed in the aperture stop 91, it is possible to acquire an image with an enlarged depth of field. Additionally, at the time of forming the light transmission region in the aperture stop 91, the light transmission region may be continuously formed from the center portion of the region toward the outer edge or may be stepwisely formed from the center portion of the region toward the outer edge.

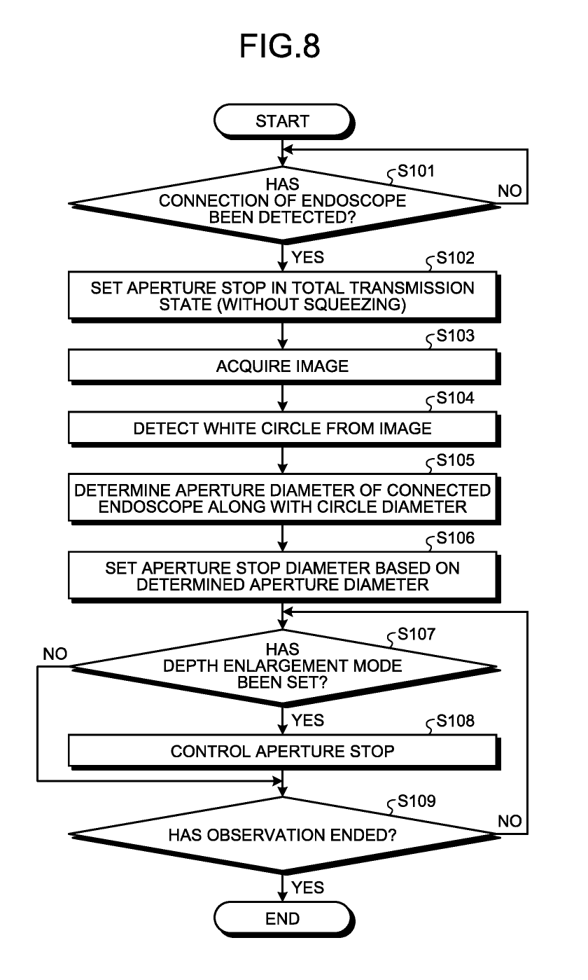

FIG. 8 is a flowchart illustrating a process that is performed by the endoscope apparatus according to the first embodiment of the disclosure. Hereinafter, a case in which the process is performed by the components under the control of the control unit 55 of the control device 5 will be described.

First, the detection unit 96 of the camera head 9 detects whether the endoscope 2 is connected (Step S101). When the connection of the endoscope 2 is not detected by the detection unit 96 (Step S101: No), the detection process is repeated by the detection unit 96. In contrast, when the connection of the endoscope 2 is detected by the detection unit 96 (Step S101: Yes), the control unit 55 moves the routine to Step S102.

In Step S102, the control unit 55 sets the aperture stop 91 into the total transmission state. Then, the control unit 55 acquires an image by the connected endoscope 2 while controlling the irradiation of the illumination light if necessary (Step S103).

In subsequent Steps S104 and S105, the aperture diameter determining unit 57 determines the aperture diameter of the optical system of the connected endoscope 2. The aperture diameter determining unit 57 detects the mask image 100 which is a white circle from the acquired image (for example, the image IM₁ illustrated in FIG. 5), in such a manner described above (Step S104). The aperture diameter determining unit 57 calculates the diameter of the detected circle (the mask image 100) and determines the aperture diameter of the optical system of the connected endoscope 2 based on the calculated diameter (Step S105).

In Step S106 subsequent to Step S105, the control unit 55 determines the aperture diameter (the aperture stop diameter) of the aperture stop 91 depending on the aperture diameter determined by the aperture diameter determining unit 57.

Then, the control unit 55 determines whether or not a depth enlargement mode is set (Step S107). The control unit 55 moves the routine to Step S108 when a signal of setting an observation mode to a depth enlargement mode is input via the input unit 54 (Step S107: Yes). In contrast, the control unit 55 moves the routine to Step S109 when the signal of setting the observation mode to the depth enlargement mode is not input via the input unit 54 (Step S107: No). Here, when the depth enlargement mode is not set, a normal observation mode is set. While the normal observation mode is set, the aperture stop 91 is kept in the total transmission state, and a normal observation is performed thereby to acquire and display a captured image through the normal observation.

In Step S108, the control unit 55 generates a control signal of setting the diameter of the light transmission region of the aperture stop 91 as a determined diameter, and outputs this control signal to the camera head controller 97. In the camera head 9, the aperture stop 91 is controlled to form a light transmission region with the determined diameter. In the aperture stop 91, the light transmission region 910 is formed which is a circle with the determined diameter. Accordingly, it is possible to obtain a captured image with an enlarged depth of field. In a state where the depth enlargement mode is set, the light transmission region 910 is formed in the aperture stop 91 to acquire and display a captured image with an enlarged depth of field.

In Step S109, the control unit 55 determines whether or not a signal of ending the observation is input via the input unit 54. When the signal of ending the observation is not input via the input unit 54 (Step S109: No), the control unit 55 moves the routine to Step S107 to continue the above-described observation process. In contrast, when the signal of ending the observation is input via the input unit 54 (Step S109: Yes), the control unit 55 ends the operation of the camera head 9 including an imaging process or the like.

In the first embodiment, while the aperture stop 91 is controlled in the total transmission state, the aperture diameter of the optical system of the connected endoscope 2 is determined based on the acquired mask image, and the diameter of the light transmission region of the aperture stop 91 in the depth enlargement mode is set from the aperture diameter. According to the first embodiment, since the diameter of the light transmission region of the aperture stop 91 is set depending on the aperture diameter of the connected endoscope 2, it is possible to generate an image with an enlarged depth of field regardless of the type of endoscope to be connected.

### Second Embodiment

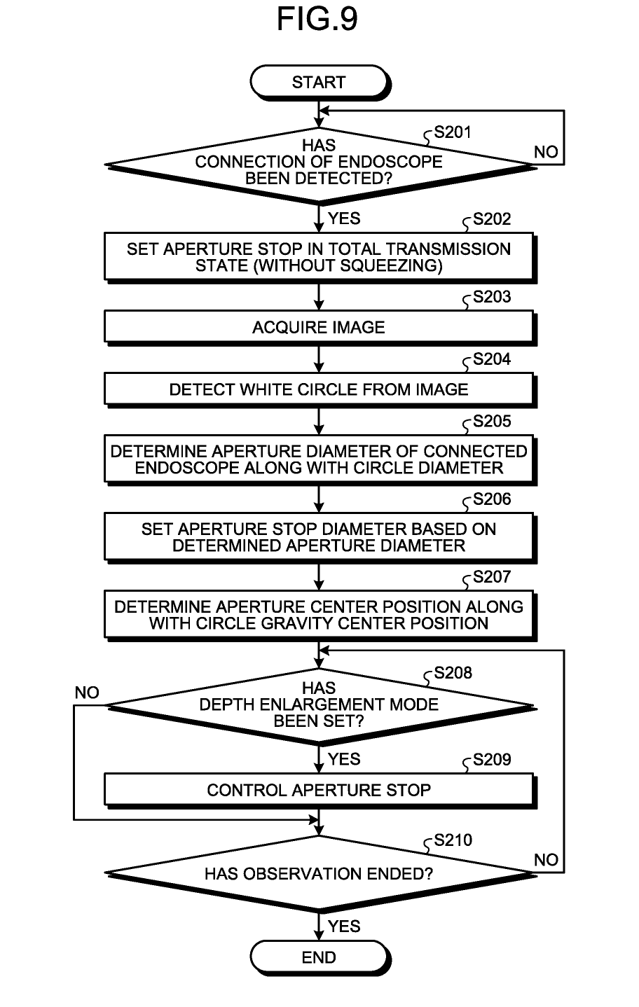

Next, a second embodiment of the disclosure will be described with reference to FIGS. 9 to 11. Since a configuration of an endoscope apparatus according to the second embodiment is the same as that of the endoscope apparatus 1, a description for the configuration will be omitted and only a process different from that of the first embodiment will be described. FIG. 9 is a flowchart illustrating a process that is performed by the endoscope apparatus according to the second embodiment of the disclosure.

Further, the detection unit 96 of the camera head 9 detects whether the endoscope 2 is connected (Step S201). When the connection of the endoscope 2 is not detected by the detection unit 96 (Step S201: No), the detection process using the detection unit 96 is repeated. In contrast, when the connection of the endoscope 2 is detected by the detection unit 96 (Step S201: Yes), the control unit 55 moves the routine to Step S202.

In Step S202, the control unit 55 sets the aperture stop 91 in the total transmission state. Then, the control unit 55 acquires an image by the connected endoscope 2 while controlling the irradiation of the illumination light if necessary (Step S203).

In subsequent Steps S204 and S205, the aperture diameter determining unit 57 determines the aperture diameter of the optical system of the connected endoscope 2.

The aperture diameter determining unit 57 detects the mask image 100 which is a white circle from the acquired image (for example, the image IM₁ illustrated in FIG. 5) (Step S204). The aperture diameter determining unit 57 calculates the diameter of the detected circle (the mask image 100) and determines the aperture diameter of the optical system of the connected endoscope 2 based on the calculated diameter (Step S205).

In Step S206 subsequent to Step S205, the control unit 55 determines the aperture diameter (the aperture stop diameter) of the aperture stop 91 in response to the aperture diameter determined by the aperture diameter determining unit 57.

Further, the control unit 55 calculates the gravity center position of the circle (the mask image 100) and determines the center position of the aperture (the light transmission region 910) of the aperture stop 91 (Step S207). In some cases, the center portion set in the image sensor of the imaging unit 93 does not match the optical axis of the optical system provided in the endoscope 2 when the endoscope 2 and the camera head 9 are connected to each other. In such a case, when the depth enlargement mode is set, the center of the mask image 100 is displaced from the center of the aperture of the aperture stop 91 so that a part of observation light is vignetted (or blocked).

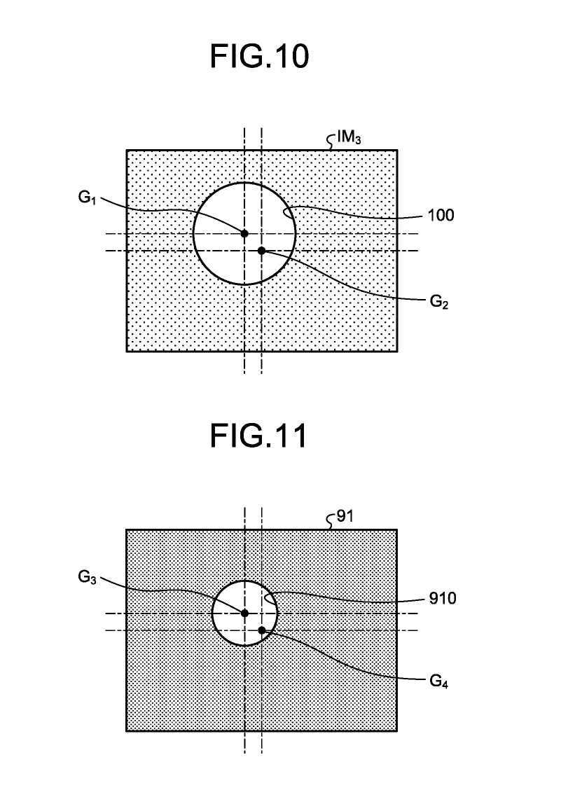

FIG. 10 is a diagram illustrating an example of an image captured by the camera head according to the second embodiment of the disclosure. When the center portion set in the image sensor of the imaging unit 93 does not match the optical axis of the optical system provided in the endoscope, the gravity center position G₁ of the mask image 100 in the captured image IM₃ and the gravity center position G₂ of the captured image IM₃ become different positions as illustrated in FIG. 10. The control unit 55 reads the coordinates of the gravity center position G₁ of the mask image 100. Corresponding coordinates are given to the captured image IM₃ and the aperture stop 91. The control unit 55 sets the coordinates of the aperture stop 91 corresponding to the gravity center position G₁ of the mask image 100 to the center position of the light transmission region 910 set in the depth enlargement mode.

By Steps S206 and S207, the position and the size of the aperture of the aperture stop 91 in the depth enlargement mode are set.

Next, the control unit 55 determines whether or not the depth enlargement mode is set (Step S208). The control unit 55 moves the routine to Step S209 when a signal of setting the observation mode to the depth enlargement mode is input via the input unit 54 (Step S208: Yes). In contrast, the control unit 55 moves the routine to Step S210 when the signal of setting the observation mode to the depth enlargement mode is not input via the input unit 54 (Step S208: No).

In Step S209, the control unit 55 generates a control signal of setting the aperture diameter of the aperture stop 91 to the determined aperture diameter, and outputs the control signal to the camera head controller 97. In the camera head 9, the aperture stop 91 is controlled to form a light transmission region with the determined diameter. In the aperture stop 91, the light transmission region 910 forming a circle with the determined diameter is formed.

FIG. 11 is a diagram illustrating the aperture stop 91 of the endoscope 2 of the second embodiment of the disclosure. When the gravity center of the mask image 100 of the captured image IM₃ is located at the gravity center position G₁, the light transmission region 910 is formed about the position (the center position G₃) corresponding to the gravity center position G₁ in the aperture stop 91 in the depth enlargement mode. Additionally, when the gravity center of the mask image 100 in the captured image IM₃ is the gravity center position G₂, the light transmission region 910 is formed about the position (the center position G₄) corresponding to the gravity center position G₂ in the depth enlargement mode.

In Step S210, the control unit 55 determines whether or not a signal of ending the observation is input via the input unit 54. The control unit 55 moves the routine to Step S208 to continue the above-described observation process when the signal of ending the observation is not input via the input unit 54 (Step S210: No). In contrast, the control unit 55 ends the operations of the camera head 9 including an imaging operation or the like when the signal of ending the observation is input via the input unit 54 (Step S210: Yes).

In the second embodiment, the aperture stop 91 is controlled in the total transmission state, the aperture diameter of the optical system of the connected endoscope 2 is determined based on the acquired mask image, and the aperture diameter of the aperture stop 91 in the depth enlargement mode is set from the aperture diameter. According to the second embodiment, because the aperture diameter of the aperture stop 91 is set depending on the aperture diameter of the connected endoscope 2, it is possible to obtain an image with an enlarged depth of field regardless of the type of endoscope to be connected.

Further, according to the second embodiment, because the center position of the light transmission region of the aperture stop 91 is determined based on the gravity center position of the mask image, it is possible to obtain a captured image with an enlarged depth of field by preventing a part of observation light from being vignetted.

Additionally, in the second embodiment, a change in center of the aperture of the endoscope 2 may be determined based on the captured image. In this case, the control unit 55 detects the motion of the subject by comparing the captured images obtained at a certain point in time and at the next point in time. The motion of the subject can be detected by a known method such as pattern matching.

Further, in the second embodiment, there are a case in which the same endoscope (for example, only the endoscope 2A) is repeatedly attached to and detached from the camera head 9, and a case in which a plurality of endoscopes having the different aperture diameters are connected to the camera head 9 as with the first embodiment. Even when the same endoscope is connected a plurality of times, the center position of the mask image may be different. As in the second embodiment, even when the same endoscope is connected, the gravity center position of the mask image at the time of connection is calculated. Then, when the center position of the light transmission region 910 is determined based on the gravity center position, it is possible to continuously acquire a captured image appropriate to enlarge the depth of field.

### Modified Example of Second Embodiment

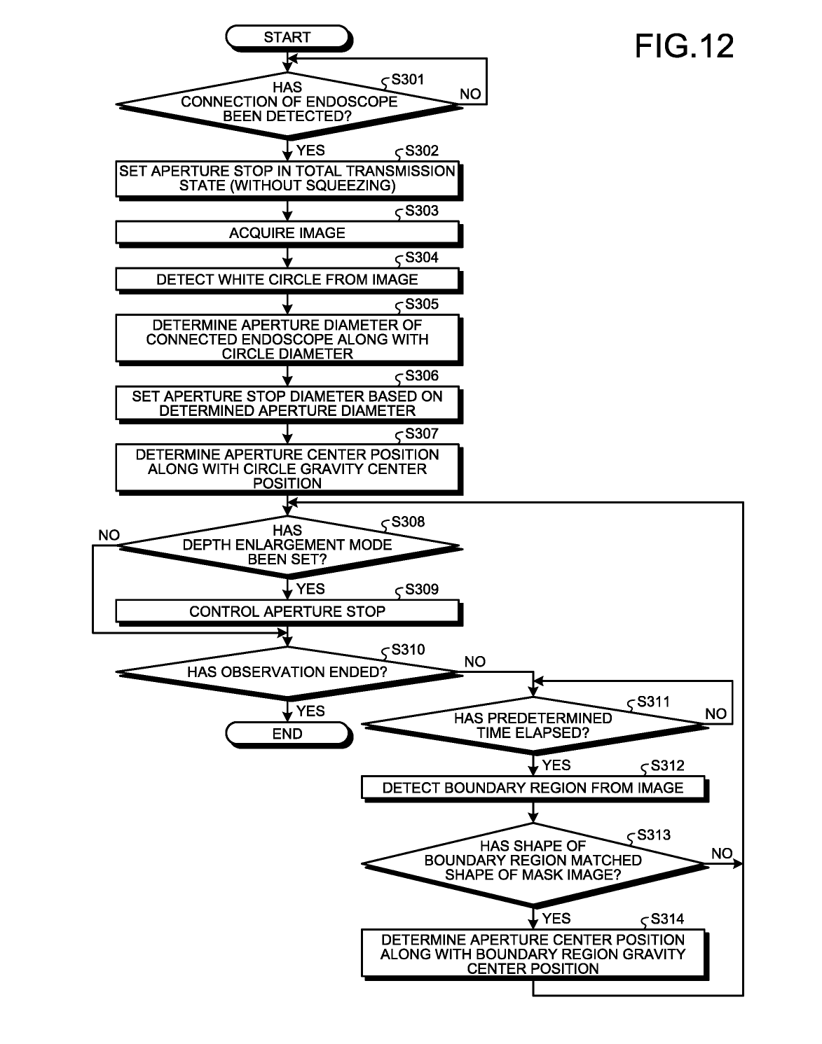

Next, a modified example of the second embodiment will be described with reference to FIG. 12. In the modified example, it is determined whether or not the position of the center of the aperture is set again at a predetermined time interval while the endoscope 2 is used in addition to a process of determining the gravity center position of the light transmission region at the time of activating the camera head 9 and connecting the endoscope 2. FIG. 12 is a flowchart illustrating a process performed by the endoscope apparatus according to the modified example of the second embodiment of the disclosure. Because Steps S301 to S309 in FIG. 12, namely, steps from the connection detection of the endoscope 2 to the camera head 9 through the control of the aperture stop 91, are the same as Step S201 to Step S209, respectively, a description thereof will be omitted.

In Step S310, the control unit 55 determines whether or not a signal of ending the observation is input via the input unit 54. The control unit 55 moves the routine to Step S311 when the signal of ending the observation is not input via the input unit 54 (Step S310: No). In contrast, the control unit 55 ends the operations of the camera head 9 including an imaging process or the like when the signal of ending the observation is input via the input unit 54 (Step S310: Yes).

In Step S311, the control unit 55 determines whether or not a predetermined time elapses from the time of determining the precedent aperture center position (Step S311). Here, the control unit 55 repeats the determination until the predetermined time elapses, when the predetermined time does not elapse (Step S311: No). In contrast, the control unit 55 moves the routine to Step S312 when it is determined that the predetermined time elapses (Step S311: Yes).

In Step S312, the control unit 55 detects a boundary region by setting a portion in which a change in luminance value is larger than a predetermined threshold value from an image acquired by the endoscope 2, for example, a recent time-series image.

Then, the control unit 55 determines whether or not a shape of the detected boundary region matches a shape of the mask image 100 (Step S313). The control unit 55 calculates a matching degree between the shape of the boundary region and the shape of the mask image 100 by using, for example, a known method such as pattern matching, and determines whether or not the two shapes match each other by comparing the matching degree with a predetermined threshold value. When it is possible to determine that the two shapes match each other as the matching degree increases, the control unit 55 determines that two shapes match each other when the matching degree is larger than a threshold value. The control unit 55 moves the routine to Step S314 when it is determined that the two shapes match each other (Step S313: Yes).

In Step S314, the control unit 55 sets the coordinates of the aperture stop 91 corresponding to the gravity center position of the boundary region to the center position of the light transmission region 910 formed in the depth enlargement mode. Then, the control unit 55 moves the routine to Step S308. In this case, an aperture (a light transmission region) having a center at a position set based on the boundary region is formed at the time of controlling the aperture stop 91 in Step S309.

Meanwhile, the control unit 55 moves the routine to Step S308 without changing the currently set aperture center position when it is determined that the two shapes do not match each other (Step S313: No). In this case, an aperture (a light transmission region) having a center at a position set based on, for example, the mask image 100 is formed at the time of controlling the aperture stop in Step S309.

In this modified example, the aperture center position of the aperture stop 91 is changed while the endoscope 2 is used. Accordingly, for example, even when a user who uses the endoscope 2 rotates the endoscope 2 with respect to the camera head 9 so that the optical axis of the endoscope 2 is displaced from the camera head 9, it is possible to prevent a part of observation light from being vignetted due to a difference in light transmission region at the time of enlarging the depth of field by the aperture stop 91. In particular, there is a case in which the endoscope 2 is rotated with respect to the camera head 9 for the observation when an oblique endoscope is used as the endoscope 2. In the oblique endoscope, the optical axis of the objective lens is inclined with respect to the longitudinal direction of the endoscope 2. Even when such an oblique endoscope is used other than the endoscope having the objective lens of the optical axis parallel to the longitudinal direction of the endoscope 2, it is possible to stably obtain a captured image at the time of enlarging the depth of field, according to this modified example.

### Third Embodiment

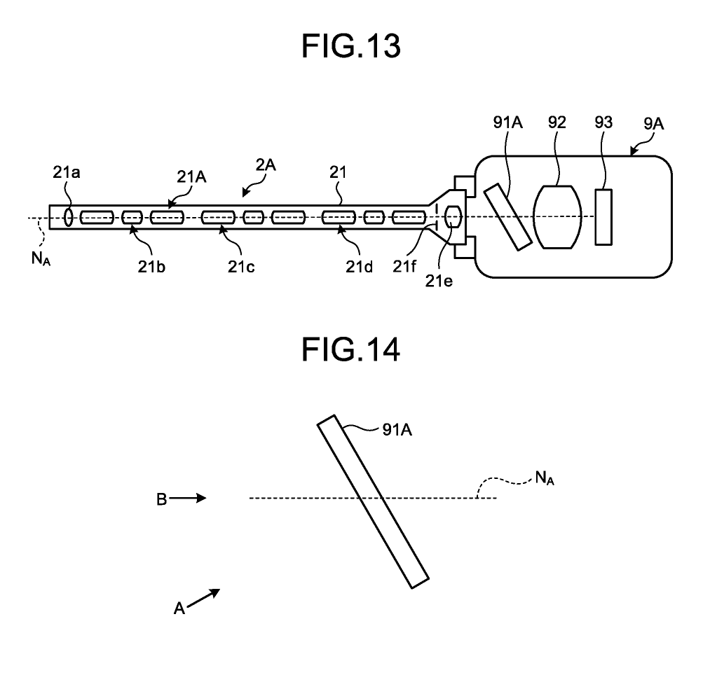

Next, a third embodiment of the disclosure will be described with reference to FIGS. 13 to 16. An endoscope apparatus according to the third embodiment has the same configuration as that of the endoscope apparatus 1 except for the configuration of the camera head. Therefore, a description other than a configuration of a camera head 9A will be omitted and only a different configuration from that of the first embodiment will be described. FIG. 13 is a schematic diagram illustrating a configuration of the endoscope and the camera head according to the third embodiment of the disclosure. FIG. 13 illustrates a configuration of the camera head 9A to which the endoscope 2A is connected, as an example.

The camera head 9A includes an aperture stop 91A, the lens unit 92, the imaging unit 93, the driving unit 94, the communication module 95, the detection unit 96, and the camera head controller 97 (see FIG. 2 for the driving unit 94, the communication module 95, the detection unit 96, and the camera head controller 97). Elements (or members) other than the aperture stop 91A are the same as those of the first embodiment. For this reason, only the configuration of the aperture stop 91A will be described below.

The aperture stop 91A is disposed at a position through which the optical axis of the camera head 9A passes and which corresponds to an entrance pupil position of the lens unit 92. Similarly to the aperture stop 91, the aperture stop 91A is formed in a plate shape in which two glass plates are bonded to each other and a liquid crystal is enclosed therein. The aperture stop 91A can change the shape, the position, and the size of the aperture by changing the orientation of the liquid crystal under the control of the driving unit 94. In the third embodiment, the optical axis N_{A} of the endoscope 2A coincides with the optical axis of the camera head 9A.

FIG. 14 is a diagram illustrating the aperture stop 91A of the endoscope 2A according to the third embodiment of the disclosure. In the aperture stop 91A, as illustrated in FIG. 14, a principal surface of the glass plate is inclined with respect to the optical axis (the optical axis N_{A}) of the camera head 9A. The principal surface mentioned herein indicates a surface having the largest area in the glass plate.

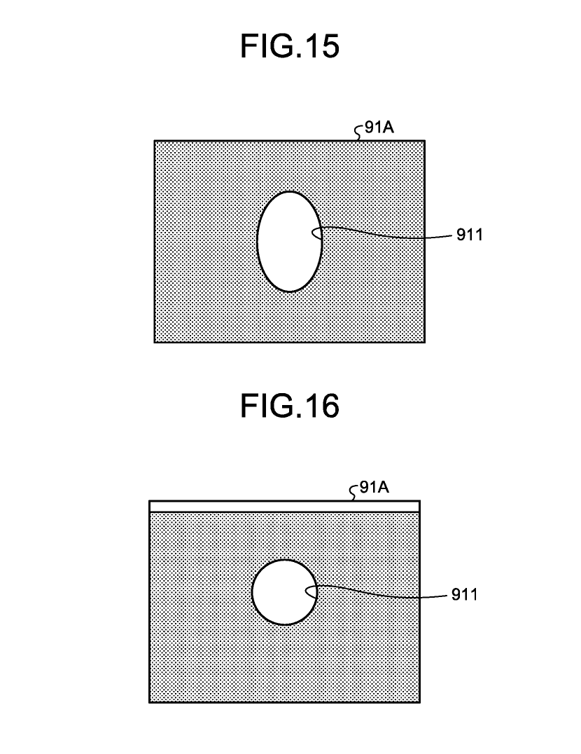

FIG. 15 is a diagram illustrating the aperture stop 91A as viewed from the direction A of FIG. 14. FIG. 16 is a diagram illustrating the aperture stop 91A as viewed from the direction B of FIG. 14. When a light transmission region having a circular shape is formed as viewed from the direction of the optical axis, the aperture stop 91A is provided with a light transmission region 911 having an oval shape (See FIG. 15) as viewed from a direction orthogonal to the principal surface of the glass plate (as viewed from the direction A). The light transmission region 911 which is formed in the oval shape has a circular shape (See FIG. 16) as viewed from the direction of the optical axis (as viewed from the direction B).

The aperture stop 91A is controlled to form a light transmission region having a circular shape as viewed from the direction of the optical axis based on the mask image acquired by the connected endoscope 2 similarly to the first embodiment. Additionally, as described in the second embodiment, the position of the light transmission region may be set based on the gravity center position of the mask image.

Here, for example, in the case of the aperture stop 91 (FIGS. 3A and 3B), the principal surface of the glass plate is orthogonal to the optical axis. In this case, the observation light reflected by the glass plate that constitutes the aperture stop 91 is reflected along the optical axis by elements or members positioned to oppose the aperture stop 91, thereby being incident onto the aperture stop 91 again (hereinafter, the light which is incident again will be referred to as returned light). When such returned light is received by the image sensor, ghost or flare appears in the captured image. The ghost is a subject image which is depicted by the returned light and the flare is a phenomenon in which white color appears on an image.

In contrast, in the third embodiment, since the principal surface of the aperture stop 91A is disposed to be inclined with respect to the optical axis, it is possible to prevent the drawbacks in which the observation light reflected by the glass plate of the aperture stop 91A is reflected in a direction different from the direction of the optical axis and the returned light is received by the image sensor. Accordingly, it is possible to prevent the ghost or flare in the captured image.

Further, in the third embodiment, it is possible to obtain the same effects as those of the first and second embodiments by performing the same process as that of the first or second embodiments. For example, the control unit 55 calculates the gravity center position of the mask image 100 and determines the center position (the position where the major axis and the minor axis intersect) of the light transmission region forming an oval shape based on the calculated gravity center position.

Incidentally, in the first to third embodiments, a case has been described in which an image of illumination light is detected from a total transmission image, thereby to distinguish an aperture diameter, and the aperture diameters of the aperture stops 91 and 91A are determined. However, embodiments of the present disclosure are not limited thereto. For example, the detection unit 96 may detect an ID or the like of the connected endoscope and may determine the aperture diameter of the aperture stop 91 in response to the detection result. At this time, the detection unit 96 is provided in, for example, the endoscope 2A and the endoscope 2B and electrically may detect an arrangement of pins having different arrangement patterns. The detection unit 96 electrically detects the pin arrangement pattern when the endoscope is connected. The detection unit 96 generates the detection information for the detected pin arrangement pattern. The control unit 55 identifies the endoscope by using the detection information. In addition, the detection unit 96 may generate the detection information by reading an IC tag or the like provided in the endoscope 2A and the endoscope 2B.

Further, in the first to third embodiments, a case has been described in which the aperture stops 91 and 91A are formed by a liquid crystal. However, embodiments of the present disclosure are not limited to the liquid crystal as long as the aperture shape can be changed. For example, an electrochromic element may be used. When the electrochromic element is used, the light transmission region to be formed is set.

Although the embodiments of the present disclosure have been described so far, the present disclosure is not limited to the above-described embodiments. In the above-described embodiments, a case has been described in which the control device 5 performs a signal process or the like, but the signal process or the like may be performed at the camera head 9.

As described above, the endoscope apparatus according to the present disclosure is useful for generating an image with an enlarged depth of field regardless of a type of endoscope to be connected.

Additional Item 1

An endoscope apparatus including:

an endoscope which includes an optical system; and

an imaging device to which the endoscope is connected and which includes an aperture stop provided with a light transmission region for allowing light to be transmitted to the connected endoscope and an imaging unit receiving the light passing through the aperture stop and converting the light into an electric signal,

wherein the aperture stop has a plate shape, and a principal surface of the aperture stop is inclined with respect to an optical axis of the imaging unit, and

wherein the light transmission region is formed such that a shape formed by an outer edge of the light transmission region is an oval shape as viewed along a direction orthogonal to the principal surface and is a circular shape as viewed along a direction of the optical axis of the imaging unit.

Additional Item 2

An endoscope apparatus including:

a first endoscope which includes a first optical system;

a second endoscope which includes a second optical system having an aperture diameter different from that of the first optical system;

an imaging device to which one of the first and second endoscopes is connected and which includes an aperture stop capable of changing the size of a light transmission region allowing light to be transmitted to the connected endoscope and an imaging unit receiving light passing through the aperture stop and converting the light into an electric signal;

an aperture diameter determining unit which determines an aperture diameter of the endoscope connected to the imaging device based on an image generated by the electric signal generated by the imaging device; and

a control unit which determines the size of the light transmission region formed by the aperture stop based on the aperture diameter determined by the aperture diameter determining unit and changes the light transmission region in the aperture stop.

According to the present disclosure, there is an effect that an image with an enlarged depth of field can be generated regardless of the type of endoscope to be connected.

Although the disclosure has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. An endoscope apparatus comprising:
an imaging device to which a plurality of kinds of endoscopes having optical systems with different aperture diameters are connectable, the imaging device including:
an aperture stop with a light transmission region that allows light to be transmitted to an endoscope among the plurality of kinds of endoscopes that is connected to the imaging device, wherein a size of the light transmission region is changeable; and
an imaging unit that receives the light transmitted through the aperture stop and converts the light into an electric signal;
an aperture diameter determining unit that determines an aperture diameter of the endoscope connected to the imaging device, based on an image generated by the electric signal generated by the imaging device; and
a control unit that determines the size of the light transmission region formed by the aperture stop, based on the aperture diameter determined by the aperture diameter determining unit, and changes the light transmission region of the aperture stop.

2. The endoscope apparatus according to claim 1,
wherein the aperture diameter determining unit determines the aperture diameter, based on an image generated by the light passing through the aperture stop by setting all regions capable of allowing the light to be transmitted or shielded to be the light transmission region.

3. The endoscope apparatus according to claim 2,
wherein a mask image obtained by light passing through a mask provided on an optical axis of the endoscope is formed on the image based on the light passing through the aperture stop, and
wherein the control unit calculates a diameter of the mask image and determines the size of the light transmission region of the aperture stop based on the calculated diameter of the mask image.

4. The endoscope apparatus according to claim 3,
wherein the control unit calculates a gravity center position of the mask image and determines a position corresponding to the calculated center position as the gravity center position of the light transmission region.

5. The endoscope apparatus according to claim 1,
wherein the aperture stop has a plate shape, and
wherein the light transmission region is formed such that a shape formed by an outer edge is a circular shape as viewed along a direction orthogonal to a principal surface of the aperture stop.

6. The endoscope apparatus according to claim 1,
wherein the aperture stop has a plate shape and a principal surface of the aperture stop is inclined with respect to an optical axis of the imaging unit, and
wherein the light transmission region is formed such that a shape formed by an outer edge of the light transmission region is an oval shape as viewed along a direction orthogonal to the principal surface and is a circular shape as viewed along the direction of the optical axis of the imaging unit.
